(19) United States
(12) Patent Application Publication
(10) Pub. No.: US 2004/0213380 A1
(43) Pub. Date: Oct. 28, 2004

(54) Method and apparatus for slot scanning digital radiography

(76) Inventors: Chris C. Shaw, Bellaire, TX (US); Xinming Liu, Houston, TX (US)

Correspondence Address: David D. Bahler, Esq., FULBRIGHT & JAWORSKI, L.L.P., Suite 2400, 600 Congress Avenue, Austin, TX, 78701, US

(21) Appl. No.: 10/830,727

(22) Filed: Apr. 23, 2004

Related U.S. Application Data
(60) Provisional application No. 60/464,790, filed on Apr. 23, 2003.

(51) Int. Cl. G21K 1/12; H05G 1/60; A61B 6/00; G01N 23/00; G21K 1/00

(52) U.S. Cl. 378/145

(57) ABSTRACT

A method includes directing an x-ray beam to a first set of image lines of an x-ray detector, resetting a first image line corresponding to the leading edge, reading a second image line corresponding to the trailing edge, moving the x-ray beam to a second set of image lines of the x-ray detector, and repeating the resetting, reading, and moving steps. An apparatus includes an x-ray generator, an x-ray detector, a readout circuit coupled to the x-ray detector, and a computing and controlling device coupled to the readout circuit, the computing and controlling device directing an x-ray fan beam to a first set of image lines of the detector, resetting a first image line, reading a second image line, moving the x-ray beam to a second set of image lines of the detector, and repeating the resetting and reading steps.

This patent application claims priority to, and incorporates by reference in its entirety, U.S. provisional patent application Serial No. 60/464,790 filed on Apr. 23, 2003, entitled, “Method and Apparatus for Slot Scanning Digital Radiography with Reduced Scattering.”

### FEDERALLY-SPONSORED RESEARCH AND DEVELOPMENT

Aspects of this invention were made with government support by National Institute of Biomedical Imaging and Bioengineering Contract/Grant No. EB00117. The government may accordingly have certain rights in this invention.

### BACKGROUND OF THE INVENTION

1. Field of the Invention

The invention relates generally to the field of radiography. More particularly, the invention relates to flat-panel digital radiography.

2. Discussion of the Related Art

Various digital radiography techniques incorporating new x-ray detector technology have been developed and investigated over the past few years. These techniques have provided methods for acquiring, displaying, storing, retrieving, distributing, processing, and analyzing digital images. The two digital radiography technologies which have been most successful in digital chest imaging applications are: computed radiography and flat-panel digital radiography.

Despite the development of new digital radiography techniques, the acceptance of scattered x-rays as part of the image signal remains a serious problem. One solution to this problem involves using an anti-scatter grid placed in front of the screen/film cassette in order to reduce the amount of scattered x-rays that reach the detector. However, this causes attenuation of a significant fraction (sometimes 40% or more) of primary x-rays. In turn, this attenuation requires an increase of the x-ray intensity by a large factor (referred to as the Bucky factor), which undesirably increases the patient dosage in order to maintain proper exposure to the film.

As an alternative to the use of anti-scatter grids, slot (or slit) scanning imaging techniques using collimated x-ray fan beams have been developed. Unfortunately, these systems commonly require the use of a heavy and bulky aft-collimator, which must be moved in alignment with the rapidly moving x-ray fan beam.

In an attempt to eliminate the need for a bulky and heavy aft-collimator, a narrowly collimated x-ray fan beam was used in conjunction with linear, solid state x-ray detectors. However, collimating the x-rays to a fan beam as narrow as the linear detectors is a difficult task, and typically results in fan beams wider than the detector width, wasted x-rays, and increased patient dose.

Another attempt to eliminate the need for a bulky and heavy aft-collimator was the use of a wider fan beam in conjunction with rectangular charge coupled devices (CCDs) operated in time delayed integration (TDI) mode, and optically coupled to an x-ray scintillator screen. But due to light loss, this method is suited for mammographic imaging applications where the field of view is small and the minification factor can be kept low, thus reducing the light losses in fiber-optical coupling. Other drawbacks of the slot scanning TDI imaging technique include the high complexity of the system, which requires accurate and precise alignment of charge transfer rate with the scanning motion of the detector assembly, and fixed fan beam width, which prevents the degree of scatter rejection and efficiency of x-ray usage to be adjusted for different imaging situations.

Yet another attempt to eliminate the need for a bulky and heavy aft-collimator was the use of an electronic collimation method using high framing rate read out. However, due to the limited image read out rate of most area digital detectors, electronic collimation requires the use of highest possible framing rate, a reduction of the image resolution, a significant increase of the scanning time, or a significant increase of the slot width. None of these compromises has been found to be acceptable for practical radiography imaging use, due to compromised noise properties from rapid image read out and the difficult task of combining the discrete slot images into one smooth image.

Thus, there is a need for providing a method and apparatus to reduce or eliminate scattered radiation in flat-panel (FP) digital radiography without attenuating primary x-rays. There is a need for a practical and efficient technique for implementing the slot scanning imaging technique. There is also a need for providing a method and apparatus for efficient electronic aft-collimation used in slot scanning imaging.

### SUMMARY OF THE INVENTION

There is a need for the following embodiments. Of course, the invention is not limited to these embodiments.

According to an aspect of the invention, a method includes directing an x-ray beam to a first set of image lines of an x-ray detector, the x-ray beam having a leading edge and a trailing edge, resetting a first image line corresponding to the leading edge, reading a second image line corresponding to the trailing edge, moving the x-ray beam to a second set of image lines of the x-ray detector, and repeating the resetting and reading steps.

According to another aspect of the invention, an apparatus includes a scanning x-ray fan beam generator, an x-ray detector having a plurality of image lines, a readout circuit coupled to the x-ray detector, and a computing and controlling device coupled to the fan beam generator and readout circuit, where the computing and controlling device embodies a computer. The computing and controlling device with the computer directs an x-ray fan beam to a first set of image lines of the x-ray detector, where the x-ray fan beam has a leading edge and a trailing edge. Further the computing and controlling device with the computer resets a first image line corresponding to the leading edge, reads a second image line corresponding to the trailing edge and moves the x-ray beam to a second set of image lines of the x-ray detector. The computing and controlling device with the computer repeats the resetting, reading, and moving steps to produce an x-ray image.

In another embodiment of the invention, a system comprising a detector comprising a plurality of image lines, a x-ray source directing an x-ray beam to a set of image lines of the plurality of image lines on the detector, a set of gate drivers coupled to the detector, and an address decoding circuit coupled to the set of gate drivers, wherein the address decoding circuit receives lines addresses corresponding to the set of image lines and activates corresponding gate drivers to reset a first image line of the set and read a second image line of the set.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

The terms a or an, as used herein, are defined as one or more than one. The terms including and/or having, as used herein, are defined as comprising (i.e., open language). The term coupled, as used herein, is defined as connected, although not necessarily directly, and not necessarily mechanically.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the nonlimiting embodiments illustrated in the drawings, wherein like reference numerals (if they occur in more than one view) designate the same or similar elements. The invention may be better understood by reference to one or more of these drawings in combination with the description presented herein. It should be noted that the features illustrated in the drawings are not necessarily drawn to scale.

FIG. 1 is a block diagram of a slot scanning digital radiography system with a digital flat-panel detector, representing an embodiment of the invention.

FIG. 2 is a block diagram of an image readout system, representing an embodiment of the invention.

FIG. 3 is a diagram of an x-ray fan beam projected onto a flat-panel x-ray detector, representing an embodiment of the invention.

FIG. 4 is a flowchart of a readout method, representing an embodiment of the invention.

FIG. 5 is a graph of a charge signal in an image pixel, illustrating an embodiment of the invention.

FIG. 6 is a graph of relative image signal profile, representing an embodiment of the invention.

### DETAILED DESCRIPTION

The invention and the various features and advantageous details thereof are explained more fully with reference to the nonlimiting embodiments that are illustrated in the accompanying drawings and detailed in the following description. It should be understood that the detailed description and the specific examples, while indicating specific embodiments of the invention, are given by way of illustration only and not by way of limitation. Various substitutions, modifications, additions and/or rearrangements within the spirit and/or scope of the underlying inventive concept will become apparent to those of ordinary skill in the art from this disclosure.

The invention may include an alternate line erasure and read out (ALER) method and apparatus for implementing electronic aft-collimation. In one embodiment, the invention may utilize a solid state or electronic detector system employing a line-by-line read out method. In another embodiment, the invention may include a method and apparatus for erasing a scatter component accumulated in an image line prior to the arrival of the scanning fan beam and readout of image signals accumulated during the fan beam exposure. The invention may be used, for example, to implement a slot scanning digital torso radiography technique, including but not limited to digital mammography or digital chest radiography. The invention may also be used for imaging the head and extremities of a subject.

In practice, the invention may utilize an amorphous silicon thin film transistor (TFT) array based flat panel (FP) detector, a charged-coupled device (CCD) based detector, or a raster scan video camera based detector. As one of ordinary skill in the art will recognize in light of this disclosure, the invention may be used with most digital or electronic x-ray image detectors employing a line-by-line read out scheme.

There are two types of amorphous silicon TFT array based flat-panel x-ray image detectors. A first type, referred to as an indirect x-ray detector, comprises an array of TFT switches and photodiodes coupled to a layer of x-ray scintillator, generally a regular x-ray phosphor screen or a layer of cesium iodide. During exposure, x-rays are absorbed and converted into light in the scintillator. The light then propagates through the scintillator and exposes the photodiodes in which charge signals are generated and stored. During image readout, the TFT switches are turned on line-by-line to transfer out the charge signals stored in the photodiodes to charge amplifiers for conversion into electronic signals, which may subsequently be digitized into digital data. In one embodiment, to increase the throughput of the readout process, each image line may be divided into a number of segments. The segmented image lines allows for a simultaneous readout of the amplified signals on a pixel-by-pixel basis through parallel channels.

A second type of amorphous silicon TFT array based flat-panel image detector, referred to as a direct x-ray detector, employs an array of TFT switches and capacitors coupled to a layer of the amorphous selenium, or other photoconductor materials. During exposure, the x-rays are absorbed/converted into electron-hole pairs in the photoconductor layer and a bias voltage (in the order of approximately 30,000 volts for amorphous selenium) may be applied across the photoconductor layer to collect the charges and store them in the capacitors. During the image readout process, the charge information stored in the capacitors may be read out by turning on the TFT switches on a line-by-line basis and transferring the charges to the charge amplifiers.

The two different types of amorphous silicon TFT array based flat-panel x-ray image detectors perform similar image readout functions, where each image line may be divided into a number of segments for simultaneous readout on a pixel-by-pixel basis through parallel channels. In most FP detectors, the main part of the image readout electronics (with timing adjusted to generate optimal combination readout time and signal-to-noise ratio) may be located approximate to the detector, while the addressing and control signals are generated by a separately located computer and electronic circuitry.

The invention may include a digital flat-panel detector with specially designed addressing and readout control electronic circuitry to synchronize image readout with the advancing fan beam and to allow the leading edge and trailing edge image lines to be alternately reset and read out as the fan beam scans across the detector. In one embodiment, the specially designed circuitry may replace all or part of the original addressing and readout control circuitry of a flat panel detector system. Alternatively, a flat panel detector system may be integrated with the addressing and readout control circuitry specially designed for this invention.

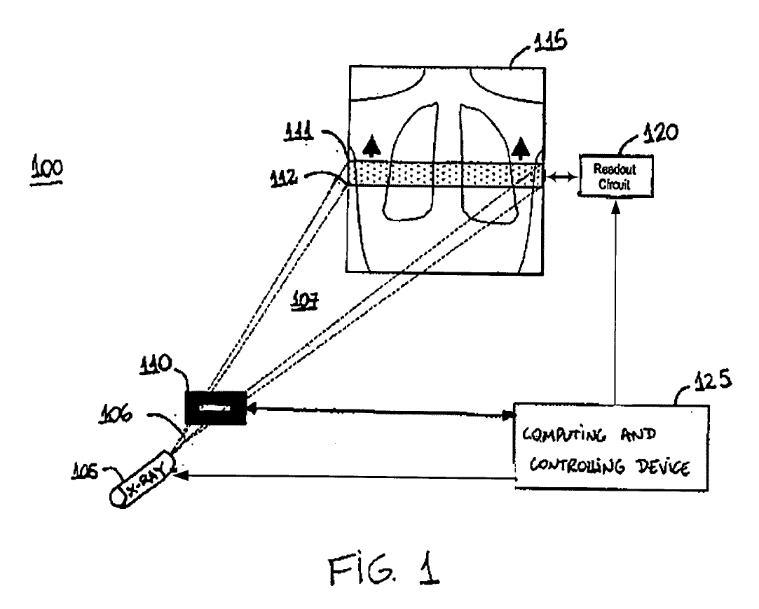

Referring to FIG. 1, a block diagram of a slot-scanning digital radiography system with a digital flat-panel detector 100 is depicted according to one embodiment of the invention. An x-ray tube assembly (source) 105 produces divergent x-rays 106 and a fore-slit collimator 110 may be used to form and move an x-ray fan beam 107 to scan the patient (not shown) positioned between the collimator 110 and detector 115. The detector 115 may be, for example, an amorphous silicon TFT array based flat-panel x-ray image detector, a charged-coupled device (CCD) based detector, a raster scan video camera based detector, or any other type of detector capable of line-by-line readout. The image lines on the leading and trailing edges 111, 112 of the fan beam may be alternately reset and transferred to a readout circuitry 120, respectively, as the fan beam 107 transmits through the patient and scans with the detector 115.

A computing and controlling device 125 may send out addressing and control signals to the readout circuit 120 to reset and read out the leading and trailing edge 111 and 112 image lines, respectively. In one embodiment of the invention, the leading edge 111 may contain scatter signals accumulated prior to the arrival of the scanning fan beam and may be discarded by transferring the charges from the photodiodes (for an indirect x-ray detector) or capacitors (for a direct x-ray detector) in the image pixels to the charge amplifiers and subsequently resetting the signals. Alternatively, the leading edge 111 image line may be read out and used to estimate the scatter component in the fan beam exposure. The results may be used to correct the fan beam exposure data obtained by reading out the trailing edge 112 image lines to further remove most of the scatter component left in these data through image analysis and processing. The computing and controlling device 125 may include, for example, a computer equipped with a multifunction data acquisition or input/output interface board. Further, the computing and controlling device 125 may include a computer program to control the operation of the readout circuit 120, the x-ray source 105, and the fore-slit collimator 110.

In practice, the fore-slit collimator 110 may include, for example, an adjustable slit width collimator and may be mounted on a translational stage driven by a computer controlled stepping motor (not shown). The fore-slit collimator 110 and the associated components may be placed between the patient and the x-ray tube assembly 105. The slit may be oriented in either horizontal or vertical direction for vertical or horizontal scanning motion, respectively. Although FIG. 1 shows a bottom-to-top scan, a top-to-bottom or a side-to-side scan are also possible within the scope of the invention. The slit width may be made adjustable and determined by the desired fan beam width as projected on the detector 115 and the geometric magnification factor of the projection. In one embodiment, the detector 115 may be oriented so that the image lines are parallel to the edges of the projected x-ray fan beam. According to another embodiment of the invention, the fore-silt collimator 110 alternately may be coupled to the x-ray tube assembly 105 such that the entire tube collimator assembly may be rotated about an axis through the focal spot to generate a scanning fan beam. The rotation may be controlled in such a way that the fan beam transmits through the patient and scans across the detector at a constant speed.

The slot-scanning digital radiography system 100 may allow the scatter component accumulated in each image line to be erased prior to the arrival and direct exposure of the scanning fan beam 107. In an initial step, the leading edge 111 image line may be reset or readout to erase the accumulated charge signal in all pixels of the line. Next, the trailing edge 112 image line may be read to acquire the signals accumulated during the fan beam exposure. Since all the trailing edge 112 image lines were erased prior to exposure to the scanning fan beam, the image formed by reading out the trailing edge 112 image lines contains mostly primary x-ray signals with only a small scatter component originating from x-rays scattered within the fan beam itself.

In one embodiment, the computing and controlling device 125 may control/monitor the position of the fore-slit collimator 110 and communicate with the read out circuit 120, thereby synchronizing the alternate erasure and readout of the leading edge 111 and trailing edge 112 image lines, respectively, to the advancing fan beam 107.

In another embodiment, the slot-scanning digital radiography system 100 may include a beam modulator used in conjunction with the fore-slit collimator 110 to regionally modulate the exposure incident to the patient to achieve more uniform exposure to the detector and thus, more uniform signal-to-noise ratio in the image, with the beam modulator also being coupled to the computing and controlling device 125. The beam modulator may include, for example, an array of pistons and elements necessary to move the pistons when controlled by the computing and controlling device 125. When using the system with a beam modulator, the invention may include acquiring a low dose unequalized image in order to determine the incident beam intensity pattern required to compensate for the unequalized attenuation pattern in the image. Next, a high dose may be applied with the pistons being positioned so as to modulate the x-ray beam intensity, thereby achieving an equalized exposure on the detector and providing an image of more uniform signal-to-noise ratio.

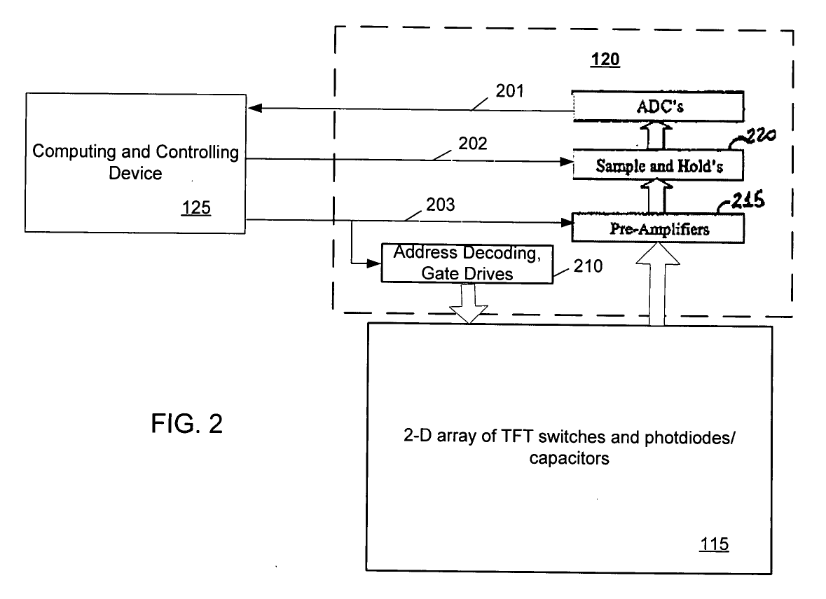

Referring to FIG. 2, a block diagram for the entire flat panel detector system 200 is depicted according to one embodiment of the invention. The entire flat panel detector system 200 may include the detector 115, the readout circuit 120, and the computing and controlling device 125 detailed in FIG. 1. The detector 115 may include a two-dimensional (2-D) array of image pixels 205, wherein the 2-D array of image pixels 205 may be organized into image lines. The 2-D array of image pixels 205 may include, for example, TFT switches and photodiodes or capacitors located underneath the scintillator or photoconductor layer. The detector 115 is coupled to the address decoding/gate driver circuit 210, the computing and controlling device 125 and to a pre-amplifier circuit 215 of the read out circuit 120. The pre-amplifier circuit 215 is coupled to a sample and hold circuit 220, and the sample-and-hold circuit 220 is coupled to an analog-to-digital converter circuit 225. In one embodiment, the pre-amplifier circuit 215 may include a plurality of pre-amplifying elements, while the sample-and-hold circuit 220 may include a plurality of sampling-and-holding elements, and the analog-to-digital converter circuit 225 may include a plurality of analog-to-digital converting elements.

In operation, the computing and controlling device 125 provides line selecting signals 203, such as address lines or clock pulses, to the address decoder/gate driver circuit 210, controlling which image line or lines are to be read out and/or reset. The computing and controlling device 125 also controls the sample-and-hold circuit 220 through a pixel address line 202 and receives a digital image data 201 from the analog-to-digital converter circuit 225.

Each line from the detector 115 may include a plurality of pixels, and each pixel may accumulate a charge signal when exposed to x-ray radiation. Further, the computing and controlling device 125 may perform a processing operation on the digital image data 201. The processing operation may include correction of the accumulated pixel charge signal for non-uniform gain or bias to create an x-ray image. The processing operation may also include re-organizing the readout signals into a slot-scan x-ray image.

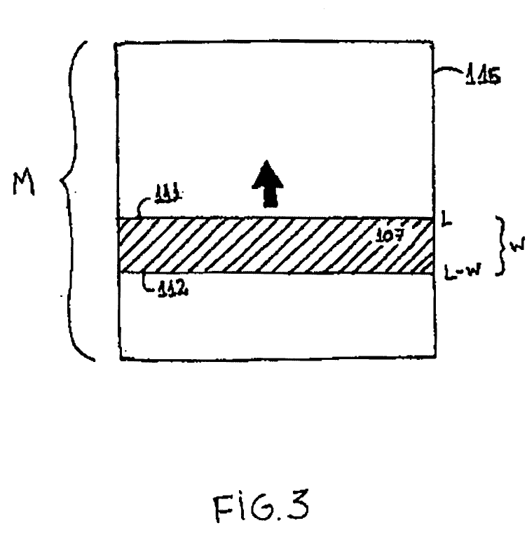

Referring to FIG. 3, a diagram of the flat-panel x-ray detector 115 detailed in FIGS. 1 and 2 is depicted according to one embodiment of the invention. The detector 115 has M image lines in the scanning direction, and the x-ray fan beam 107 is W lines wide in the scanning direction. During a scanning operation, if the leading edge 111 is at line L, the trailing edge 112 is at the line L-W.

In one example, the detector 115 may have a pixel size (pitch) of 200 μm and the x-ray fan beam 107 may have a width of 2 cm. In that case, beam 107 may be 100 lines wide (W=100). If the scan is to be completed across a length of 42 cm, then up to 2100 (42 cm/200 μm=2100) image lines may be reset to erase the scatter signals just before the fan beam arrives. The same number of image lines may be read out just after the fan beam passes to form the slot-scan x-ray image with a lowered scatter fraction.

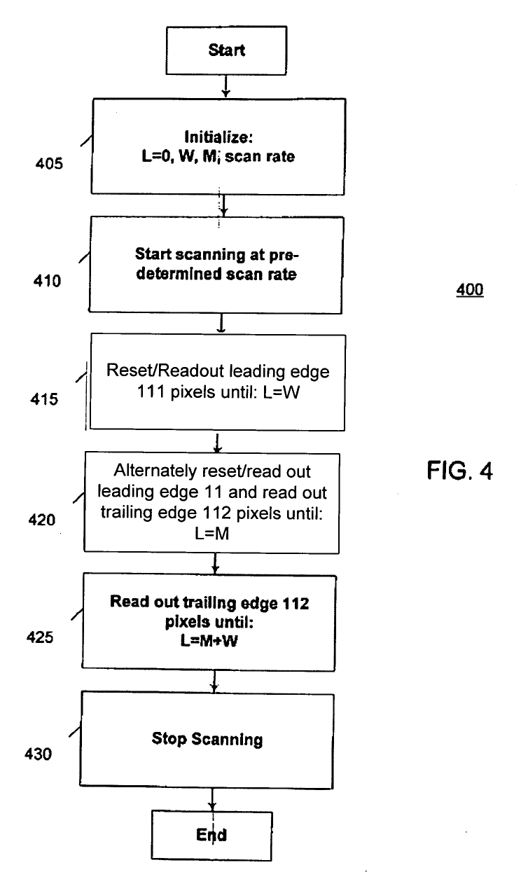

Referring to FIG. 4, a flowchart of a readout method 400 is depicted according to one embodiment of the invention. The readout method 400 may be implemented via the readout circuit 120 and the computing and controlling device 125 of the slot-scanning digital radiography system 100 detailed in FIG. 1. In an initialization step 405, the leading edge 111 image line index, L, is set to zero, and the variables, W and M, are chosen or determined as detailed in FIG. 3. Additionally, a scan rate of the x-ray fan beam 107 may be selected. In step 410, a scanning operation starts at the pre-selected scan rate.

In step 415, the leading edge 111 image line is reset until the leading edge 111 of the scanning fan beam reaches line W (L=W). Prior to the end of this step, the trailing edge 112 lies outside the detector (L-W<0) and is not read out. In step 420, the leading edge and trailing edge 111, 112 image lines are alternately reset and readout, respectively until the leading edge 111 of the scanning fan beam reaches the last line of the image (L=M). In step 425, only the trailing edge 112 image lines are read out until the leading edge 111 of the scanning fan beam passes the last line of the image by W lines (L=M+W). In step 430, the slot scanning operation ends.

The resetting of the leading edge 111 image lines during step 415 (when only part of the x-ray fan beam 107 enters and covers the detector 115) serves to erase the scatter signals accumulated prior to arrival of the x-ray fan beam. The readout of the trailing edge 112 image lines during the step 425 (after the x-ray fan beam 107 begins leaving the detector 115) results in data stored and processed to become a part of the x-ray image.

In one embodiment, the leading edge 111 image lines may be reset without reading out the scatter signals during the alternate resetting/reading process of step 420, thus achieving the erasure of the scatter signal accumulated prior to the arrival and direct exposure of the scanning fan beam. Meanwhile, the trailing edge 112 image lines read out may be stored in the computing and controlling device 125 and then processed to form a slot-scan x-ray image.

In another embodiment, both the leading edge 111 and trailing edge 112 image lines may be read out and stored in the computing and controlling device 125 during the alternate erasure and readout process of step 420. Next, the signals in leading edge 111 image lines may be used to estimate the scatter component in the fan beam exposure data which may be subtracted from the trailing edge 112 image data to mathematically remove most of the scatter component left in the slot-scan x-ray image.

The invention may include reading at least one leading edge 111 and trailing edge 112 image lines. In one embodiment, multiple leading edge 111 and/or trailing edge 112 image lines may be read out and processed by the computing and controlling device 125 in order to further reduce the scatter component in the slot-scan x-ray image.

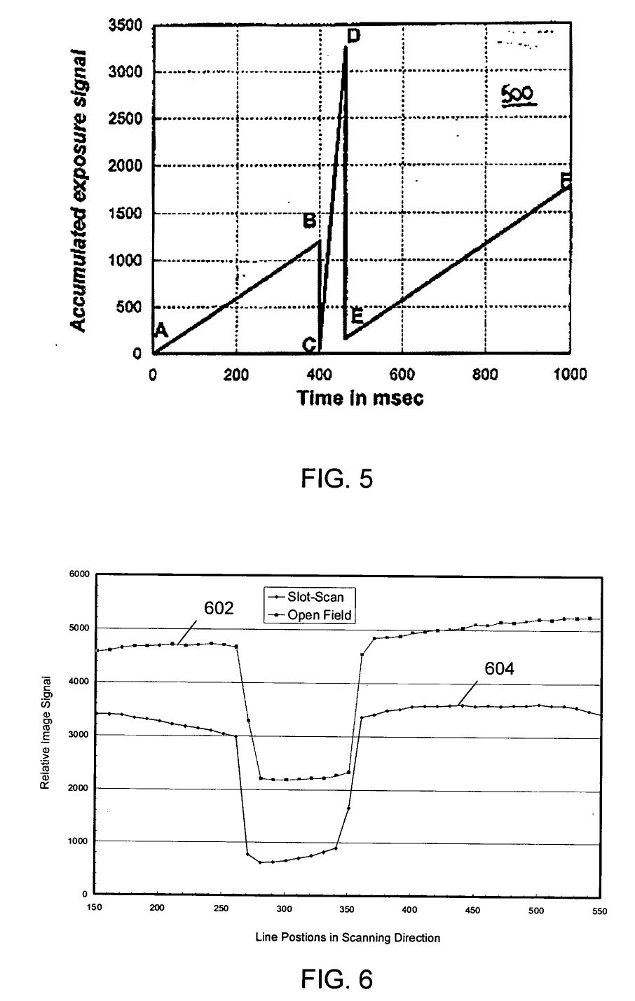

Referring to FIG. 5, a graph of a charge signal 500 in an image pixel is depicted illustrating an embodiment of the invention. An image pixel is a sub-unit of the detector 115 of FIG. 1. Graph 500 shows the temporal change of the charge signal in an image pixel during the slot scanning imaging process that has been simulated and computed using a mathematical model. The horizontal axis is the time in milliseconds, and the vertical axis is the accumulated exposure (charge) signal.

Segment A-B shows the accumulation of the scatter signal prior to arrival and direct exposure of the x-ray fan beam 107. Segment B-C shows the erasure of the scatter signal accumulated in Segment A-B right before the arrival and direct exposure of the x-ray fan beam 107. Segment C-D shows the accumulation of the fan beam exposure signal as the fan beam passes the pixel. Segment D-E shows that the accumulated fan beam exposure signal is read out right after the trailing edge 112 of the fan beam 107 passes the pixel. Segment E-F shows that following the signal readout, the charge signal accumulates again due to x-rays scatter from the passed fan beam. However, the largely scatter free fan beam exposure signal has been read out during segment D-E.

The invention may allow reducing the maximum fan beam width in a slot-scanning digital radiography apparatus. Due to the increased detective quantum efficiency (DQE) inherent of some flat-panel detectors (70% versus 25% for computer radiography systems) and removal of the anti-scatter grid (detector exposure increased by approximately 70% with chest imaging techniques), the exposure level may be reduced by a factor of at least approximately 4 while maintaining the same image signal to noise ratio (SNR). This reduction may be achieved by narrowing the overall slit width (without increasing x-ray tube output or prolonging exposure time), resulting in more effective scatter reduction, improved low contrast performance in heavily attenuated regions, and lowered patient exposure.

### EXAMPLES

Specific embodiments of the invention will now be further described by the following, nonlimiting examples which will serve to illustrate in some detail various features. The following examples are included to facilitate an understanding of ways in which the invention may be practiced. It should be appreciated that the examples which follow represent embodiments discovered to function well in the practice of the invention, and thus can be considered to constitute preferred modes for the practice of the invention. However, it should be appreciated that many changes can be made in the embodiments which are disclosed while still obtaining like or similar result without departing from the spirit and scope of the invention. Accordingly, the examples should not be construed as limiting the scope of the invention.

By using the ALER technique as described above, there is a significant reduction in the scatter-to-primary ratio (SPR). For example, a lead bar was placed in the middle of the field in the direction of the image lines. Using an open field exposure without using a scatter rejection technique, a signal profile 602 was charted to assess the effectiveness of the scatter rejection, as shown in FIG. 6. A signal profile 604 of a slot-scanning imaging using a flat panel detector, such as an a-Se flat panel detector, and the ALER technique was also assessed. A motor driven fore-collimator was mounted on an x-ray tube to generate a scanning fan beam with a width set of approximately 2 cm. The scanning speed and magnification were adjusted to synchronize the fan beam motion with the image line readout rate. As illustrated in FIG. 6, the use of slot scanning with the ALER technique resulted in a reduction of the scatter signals (as measured by the signals in the lead bar region) relative to the total signal (as measured by the signals outside the lead bar region). As a result, the scatter fraction (the ratio of the scatter component to the total signal) decreased from 44% to 16% and the scatter-to-primary ratio decreased from 79% to 19%. In another example, by using a narrower slot width, the scatter fraction and the scatter-to-primary ratio may further be reduced.

All of the methods and apparatuses disclosed and claimed herein can be made and executed without undue experimentation in light of the present disclosure. While methods and apparatuses of this invention have been described in terms of preferred embodiments, it will be apparent to those of skill in the art that variations may be applied to the methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method, comprising:
directing an x-ray beam to a first set of image lines of an x-ray detector, the x-ray beam having a leading edge and a trailing edge;
resetting a first image line corresponding to the leading edge;
reading a second image line corresponding to the trailing edge;
moving the x-ray beam to a second set of image lines of the x-ray detector; and
repeating the resetting, reading, and moving steps to provide an x-ray image.

2. The method of claim 1, wherein directing the x-ray beam to the first set of image lines comprises controlling an x-ray source.

3. The method of claim 1, wherein directing the x-ray beam to the first set of image lines comprises controlling a fore-collimator.

4. The method of claim 1, wherein directing the x-ray beam to the first set of image lines comprises controlling a modulator.

5. The method of claim 1, wherein resetting the first image line comprises reading and discarding at least one accumulated pixel charge signal.

6. The method of claim 1, wherein resetting the first image line comprises erasing a scatter signal.

7. The method of claim 1, further comprising using the second image line information to create the x-ray image.

8. The method of claim 7, wherein using the second image line information to create the x-ray image includes combining at least one accumulated pixel charge signal.

9. A method, comprising:
directing an x-ray beam to a first set of image lines of an x-ray detector, the x-ray beam having a leading edge and a trailing edge;
reading a first image line corresponding to the leading edge;
reading a second image line corresponding to the trailing edge;
moving the x-ray beam to a second set of image lines of the x-ray detector;
repeating the reading and moving steps; and
processing the first and second image lines to compensate for x-ray scattering.

10. The method of claim 9, further comprising using data from the first image line to estimate and remove a scatter information from the second image line.

11. The method of claim 10, wherein using the first image line to remove a scatter information from the second image line comprises subtracting a scatter signal estimated from a first image line pixel value from a second image line pixel value.

12. An apparatus, comprising:
a scanning x-ray fan beam generator;
an x-ray detector having a plurality of image lines;
a readout circuit coupled to the x-ray detector; and
a computing and controlling device coupled to the fan beam generator and readout circuit, the computing and controlling device embodying a computer which, at least:
directs an x-ray fan beam to a first set of image lines of the x-ray detector, the x-ray fan beam having a leading edge and a trailing edge;
resets a first image line corresponding to the leading edge;
reads a second image line corresponding to the trailing edge;
moves the x-ray beam to a second set of image lines of the x-ray detector; and
repeats the resetting, reading, and moving steps to produce an x-ray image.

13. The apparatus of claim 12, the x-ray beam generator comprising:
an x-ray source coupled to the computing and controlling device for producing divergent x-rays; and
a fore-slit collimator coupled to the x-ray source and to the computing and controlling device for producing a scanning x-ray fan beam.

14. The apparatus of claim 13, the fore-slit collimator further comprising an adjustable slit width fore-slit collimator.

15. The apparatus of claim 13, the fore-slit collimator and the x-ray source being operably rotatable by a motor coupled to the computing and controlling device.

16. The apparatus of claim 13, further comprising a motor coupled to the fore-slit collimator and to the computing and controlling device.

17. The apparatus of claim 12, the x-ray detector comprising a digital x-ray detector for reading out line-by-line image data.

18. The apparatus of claim 17, the digital x-ray detector comprising an amorphous silicon thin film transistor array based flat panel detector.

19. The apparatus of claim 17, the digital x-ray detector comprising a charged coupled device based detector.

20. The apparatus of claim 17, the digital x-ray detector comprising a raster scan video camera based detector.

21. A system, comprising:
a detector comprising a plurality of image lines;
a x-ray source directing an x-ray beam to a set of image lines of the plurality of image lines on the detector;
a set of gate drivers coupled to the detector; and
an address decoding circuit coupled to the set of gate drivers, wherein the address decoding circuit receives line selecting signals corresponding to the set of image lines and activates corresponding gate drivers to reset a first image line of the set and read a second image line of the set.

22. The system of claim 21, the detector comprising a digital x-ray detector.

23. The system of claim 22, the digital x-ray detector comprising a two-dimensional array of thin film transistor switches and capacitors.

24. The system of claim 22, the digital x-ray detector comprising a two-dimensional array of thin film transistor switches and photodiodes.

25. The system of claim 21, the line selecting signals comprising line addresses.

26. The system of claim 21, the line selecting signals comprising clock pulses.
